(19) United States
(12) Patent Application Publication
(10) Pub. No.: US 2001/0026813 A1
(43) Pub. Date: Oct. 4, 2001

(54) Extract from scutellariae radix having neuroprotective effects and pharmaceutical preparations containing the same

(76) Inventors: Ho-Cheol Kim, Seoul (KR); Duk-Kyun Ahn, Seoul (KR); Sun-Yeou Kim, Kyunggi-do (KR); Kyungho Suk, Seoul (KR); Young-Ok Kim, Seoul (KR); Kang-Hyun Leem, Seoul (KR)

Correspondence Address: ABELMAN FRAYNE & SCHWAB, Attorneys at Law, 150 East 42nd Street, New York, NY, 10017, US

(21) Appl. No.: 09/777,630

(22) Filed: Feb. 6, 2001

(30) Foreign Application Priority Data
Feb. 10, 2000 (KR) 2000-6310

(51) Int. Cl. A61K 35/78

(52) U.S. Cl. 424/741

(57) ABSTRACT

Disclosed are a Scutellariae Radix extract and a pharmaceutical preparation comprising the extract as a pharmaceutically effective ingredient. With significant neuroprotective activity, but no toxicity, the Scutellariae Radix extract is suitable for use in the prophylaxis and treatment of brain diseases, such as apoplexy, Parkinson's disease and senile dementia.

### BACKGROUND OF THE INVENTION

The present invention relates to an extract from Scutellariae Radix, which is of neuroprotective activity and a pharmaceutical preparation comprising the Scutellariae Radix as a pharmaceutically effective ingredient, suitable for use in the prophylaxis and treatment of brain diseases.

Scutellariae Radix, which is pharmaceutically useful in Oriental medicine, is obtained by barking and drying roots of Scutellaria baicalensis Georgi, a perennial herb belonging to Labiatae. It is known that Scutellariae Radix contains a variety of flavonoids, such as wogonin, baicalin and baicalein.

In Oriental medicine, the plant has conventionally been used for wet-hear removal, hemostasis, and fetus settlement. Recently, its various pharmaceutical activities have been newly disclosed, including antibacterial activity, anti-inflammatory activity (Michinori Kubo et al., Chem. Pharm. Bull., 32(7), 1984), anti-allergic activity, bile secretion promoting activity, liver protection activity, diuresis, hyperlipidemia remedy, intestinal motility inhibitory activity, and anti-cancer activity (State Administration of Traditional Chinese Medicine; Chinese Pharmacopoeia, Shanghai, Shanghai Science & Technology Press, 1998, pp1682-1694). Clinically, Scutellariae Radix has been used in various prescriptions for curing hypertension, epidemic cerebro-spinal meningitis, mild paralysis, etc. (Kim, et al., Prescription, Young Lim Publications, Seoul, 1990, pp111-113, pp263-264, p338). For example, Scurellariae Radix is contained in a Coptidis Rhizoma-based medicine in draught for counteracting poisonous effects, a Gentiana Scabra Bunge. Var. buergeri Max.-based medicine in draught for activating functions of the liver, an ox benzoar-based pill for activating the heart, and an Ostericum koreanum (Maximowz) Kitagawa-based medicine in draught for treating paralysis and an ox benzoar-based pill or preventing a sudden heart failure.

### SUMMARY OF THE INVENTION

In the present invention, Scutellariae Radix, which is a main component of an Ostericum koreanum (Maximowz) Kitagawa-based medicine in draught for treating paralysis and an ox benzoar-based pill for preventing a sudden heart failure, is examined for its protective activity against the damage of neuronal cells. Thus far, there have not yet been developed chemical medicines effective for the treatment of paralysis in the early stage.

With the aim of suggesting an effective cure for early-stage paralysis on the basis of Oriental medicine, which has accumulated a large quantity of data, the present inventors have conducted intensive and thorough research on the pharmaceutical effects of Scutellariae Radix on early-stage paralysis, and its therapeutic mechanism, and finally found that an extract from Scutellariae Radix has protective activity against the damage of neuronal cells.

Therefore, it is an object of the present invention to provide a Scutellariae Radix extract which shows neuroprotective activity.

It is another object of the present invention to provide a pharmaceutical preparation which is therapeutically effective for the prophylaxis and treatment of brain diseases associated with neuronal damage.

In accordance with an aspect of the present invention, there is provided a Scutellariae Radix extract with neuroprotective activity, prepared by soaking Scutellariae Radix in an extractant, said extractant being selected from water, low alcohols, and mixtures thereof, concentrating the extractant, and freeze-drying the concentrate.

In accordance with another aspect of the present invention, there is provided a pharmaceutical preparation with neuroprotective activity, comprising a Scutellariae Radix extract as a pharmaceutically effective ingredient in combination with a pharmaceutically acceptable base, said Scutellariae Radix extract being prepared by soaking Scutellariae Radix in an extractant selected from water, longs alcohols, and mixtures thereof, concentrating the extractant, and freeze-drying the concentrate.

### BRIEF DESCRIPTION OF THE DRAWINGS

FIG. 1 shows curves in which, after the induction of ischemia and the completion of re-perfusion in rats, the body temperature is plotted against time for injection doses of the Scutellariae Radix extract when the body temperature uncontrolled (a) and externally controlled (b).

FIG. 2 shows microphotographs of the light hippocampus of rats on the 7^{th} day after the induction of ischemia for 10 min in mock groups (a and b), a control group (c) and Scutellariae Radix-treated groups (d, e and f).

FIG. 3 is a histogram showing the neuroprotective effects of the Scutellariae Radix extract in a dose-dependent pattern 7 days after the induction of ischemia for 10 min.

FIG. 4 shows histograms in which the antioxidative activity of the Scutellariae Radix extract is measured by an MTT assay (a) and an LDH assay (b).

### DETAILED DESCRIPTION OF THE INVENTION

Scutellariae Radix was tested for its therapeutic effect on paralysis in the 4-vessel occlusion model, which was developed by Pulsinelli, 1979. In this model, representative of the animal models of forebrain ischemia, vessels through which blood is provided to the brain of a rat are occluded temporarily and re-perfused to bring about damage to neuronal cells in the area of the hippocampus. The neuronal cell damage is of natural cell necrosis, whose progress follows an apoptotic pathway. Drugs which are therapeutically useful in this model are known to exhibit curative effects on all animal models suffering from local ischemia like human paralysis. In fact, the drugs have shown clinically useful potential. For the study of ischemic neuronal cell damage, animal models suffering from the forebrain ischemia caused by 4-vessel occlusion have recently been preferred to animal models suffering from complete forebrain ischemia because blood currents in the hind-brain are not affected upon 4-vessel occlusion so that the influence of breathing and systemic circulation on the study is excluded.

In the present invention, the protective effects of Scutellariae Radix versus the neuronal cell damage caused by cerebral ischemia is described. To this end, an extract from Scutellariae Radix is injected immediately after the induction of cerebral ischemia and, one week after the injection, viable neuronal cells in the CA1 subfield of the hippocampus are counted to determine, whether Scutellariae Radix is effective for the treatment of paralysis caused by the apoptosis of neurons. Also, Scutellariae Radix is examined for antioxidative activity to study the neuroprotective mechanism thereof. In this regard, the antioxidative activity is observed in vitro for a PC12 cell line which is cultured and oxidatively damaged by hydrogen peroxide. In this model, because the cell damage is known to be associated with the induction of a group of caspases which play a role in the apoptotic pathway, a immunohistochemistry is used to determine whether Scutellariae Radix has inhibitory activity against the induction of caspase 3 (cpp32), which plays a major role in the enzymatic action of the caspase group.

For use in such experiments, an extract is obtained from Scutellariae Radix by use of a solvent extraction. As a solvent suitable for this extraction, there can be used water, low alcohols, and mixtures thereof. Examples of the lower alcohols include methanol, ethanol, n-propyl alcohol, isopropyl alcohol, n-butyl alcohol and t-butyl alcohol, with preference for methanol and ethanol. To obtain an extract, Scutellariae Radix is soaked in the solvent and the extract is filtered, concentrated and freeze-dried.

A better understanding of the present invention may be obtained in light of the following examples which are set forth to illustrate, but are not to be construed to limit the present invention.

### EXAMPLE 1

### Preparation of Extract from Scutellariae Radix

3 kg of dried Scutellarie Radix was sonicated for 15 min three times in a 70% methanol aqueous solution. After being pooled together, the extract was filtered and concentrated under vacuum, followed by freeze-drying the concentrate to give 160 g of a freeze-dried extract.

### EXAMPLE 2

3 kg of dried Scutellarie Radix was sonicated for 15 min three times in distilled water. After being pooled together, the extract was filtered and concentrated under vacuum, followed by freeze-drying the concentrate to give 150 g of a freeze-dried extract.

### EXAMPLE 3

3 kg of dried Scutellarie Radix was sonicated for 15 min three times in a first grade ethanol. After being pooled together, the extract was filtered and concentrated under vacuum, followed by freeze-drying the concentrate to give 1450 g of a freeze-dried extract.

### EXPERIMENTAL EXAMPLE

Experimental Animal

5-week old Wister female white rats with a body weight of about 170 g (SLC, Japan) were adapted to the laboratory environment with free access to feedstuffs and water for one week before testing.

Materials

Scutellariae Radix was certified by the Laboratory of Herbology, College of Oriental Medicine, Kyung Hee University, Korea, before testing.

Ischemia Induction

After being put under anesthesia, a Wister rat was laid on its back in a stereotaxic apparatus in such a manner that the tail was fixedly directed at a downward angle of 30° on the horizontal die of the apparatus while the nose and the mouth were fitted into a plastic cone connected to an anesthesia device (Ohameda V.M.C./Boc Health Care, Cyprane, U.K.). The anesthetization was first achieved by use of 5% isoflurane in a mixture of nitrogen and oxygen (N₂ 70% and O₂ 30%) and then, maintained with 1.5% isoflurane.

The tail was fixed on an operating table while the cervical vertebra was extended. First, the throat region was opened. Following that, silicon tube rings were established in common carotid arteries to cause ischemia with a design capable of performing re-perfusion. To block blood circulation through microvessels when causing the ischemia, a thread was passed through the rat's body in such a way that cervical and paraverterbral muscles were positioned, ahead of the trachea, the esophagus, the external jugular vein, and the common carotid arteries, on the thread, after which the wounds were sutured with operating clips.

Next, the rat was laid on its stomach for surgery on the occipital bone portion. With the aid of an operating magnifier, the first cervical vertebral position below the occipital bone was operated on. A micro electrocautery needle with a size of 1 mm or less approached the alar foramina with care being taken to avoid damaging muscles, and put through the alar foramina of the first cervical vertebra, into the tunnel through which the vertebral artery runs. The vertebral artery was electrically cauterized by flowing an electric current to the needle intermittently. After confirmation of the complete electrocauterization and occlusion of the vertebral artery running through the tunnel in the spine by use of an operating microscope, suturing was conducted with operating clips. After 24 hours, the operating clips were removed. Subsequently, common carotid arteries were occluded for 10 min with aneurysm clips to cause ischemia. If a light reflex vanished within 1 min, the cervical portion was further tightly sutured. Rats which did not show the disappearance of the light reflex in spite of the tightened sutures were excluded from the experiment because they were judged to have undergone complete, parallel damage on opposite sides of the CA1 neuron. Those which fell into convulsions were also excluded. After 10 min, the aneurysm clips were removed from the common carotid arteries, followed by the re-perfusion of the arteries. Only those rats which showed a consciousness loss period of 20±5 min after the re-perfusion were chosen for further study.

The rat was monitored for body temperature for 6 hours at intervals of 30 min after the induction of the ischemia. Where the body temperature decreased, the change in the body temperature was recorded while the body temperature was not controlled. In another experiment, the body temperature decrease was blocked so that the defensive effect attributable to low body temperature on neuronal cells was excluded. In this case, an automatic temperature controller, which can take advantage of the temperature of the rectum, was employed to maintain the body temperature of the rats at 37±0.5° C. during the induction of ischemia, the re-perfusion and recovery period. The body temperature was measured with a probe which was inserted into the rectum to a length of at least 6 cm because the temperature of the rectum reflects that of the brain (Miyazawa T et al., J. Cereb Blood Flow Metab 1992: 12: 817-822).

Administration of Herbal Sample and Selection of Experiment Group

In order to measure the therapeutic effect of Scutellariae Radix on forebrain ischemia in rats, extracts from the herb were administered at different doses to the rats. The administration of the extract was conducted at 0 and 90 min after the induction of the forebrain ischemia. The Scutellariae Radix extracts prepared in the above examples were dissolved in 0.89% saline, and intraperitoneally injected at doses of 250 mg, 500 mg and 1,000 mg of the component per kg of body weight with the same volume of injections. As a mock group, the first group underwent a surgical operation in the same manner, but forebrain ischemia was not induced. To a second group, which served as a control group, a physiological saline was intraperitoneally injected at a dose of 2.0 ml/kg at the same time intervals as the administration of the herbal extract after the forebrain ischemia was induced in the group in the same manner. The Scutellariae Radix extracts were intraperitoneally injected at doses of 250 mg/kg, 500 mg/kg and 1,000 mg/kg to a third, a fourth and a fifth group, respectively, at 0 and 90 min after the induction of forebrain ischemia.

Preparation of Tissue Specimen

One week after the induction of forebrain ischemia, the rats were anesthetized with chloral hydrate (35.0 mg/kg i.p.) and their chests were opened. The right auricle was cut open and a syringe was carefully inserted into the left ventricle, after which heparinized, 0.5% sodium nitrite physiological saline was slowly but constantly perfused into the heart and then, a 4.0% formalin fixative was used for the heart perfusion. Thereafter, the brain was removed and subjected to postfixation for 2 hours in a 0.1 M phosphate-buffered formalin fixative, followed by infusion with 30% sucrose at 4° C. overnight. From the fixed brain, a coronal block of a dorsal hippocampus portion extending between −2.5 mm and −4.0 mm from the bregma point was prepared. Next, the coronal block was cryosectioned using a sliding microtome. Hippocampal tissue sections with a thickness of 30 μm were collected for sample preparation.

Observation of Damaged Neurons

After the tissue sections comprising the dorsal hippocampus were dyed with cresyl violet and fixed, neuronal cells were counted in the 1,000 μm-long middle zone, which is the most susceptible to delayed neuronal death in the CA1 of the dordal hippocampus (Crain B. J. et al., Neuroscience 1988; 27:387-402). Under 250 power magnification, he number of neuronal cells was determined by taking the mean of pyramidal cell counts with normal morphology in left and right sides of three different sections of the brain tissue, that is, in 6 portions in total, by three different observers.

Cultivation of PC12 Cells and Determination of Antioxidative Effect

In a 96-well plate, each well containing DMEM (Dulbecco's Modified Eagle Medium, Gibco BRL, U.S.A.) added with 1% penicillin-streptomycin (Gibco BRL, U.S.A.) and 10% fetal bovine serum (Gibco BRL, U.S.A.), PC12 cells (Rat pheochromocytoma line), purchased from the Korean Cell Line Bank, were cultured at a cell density of 3×10⁴ cells/well overnight at 37° C. in an incubator. A Scutellarie Radix extract was dissolved in a 10% DMSO (dimethyl sulfoxide, Sigma U.S.A.) solution in DPBS (Dulbecco's phosphate buffered saline, Sigma U.S.A.) to give solutions comprising the extract at a final concentration of 10.0, 25.0, 50.0 and 100.0 μg/ml. The cells were pretreated with these solutions for three hours. For comparison, the same volume of a solution of 10% DMSO in DABS was added as a control. In the media, DMSO was contained in a final concentration of 0.5%. After three hours of the pretreatment, the extract-added media was replaced by fresh media containing 0.5 mM H₂O₂ and incubation was conducted at 37° C. for 24 hours.

For assaying LDH (lactose dehydrogenase) activity, 30 μl of the medium was transferred into each well of a new 96-well plate, which was then added with 30 μl of a solution containing 0.75 mM pyruvate and 1.4 mM NADH, followed by incubation at 37° C. for 30 min. Thereafter, a coloring solution (2,4-dinitrophenylhydrazine, Young Dong, Korea) was reacted with the medium and alkalinized with 0.4 N NaOH to express colors, which were measured for absorbance at 405 nm in a microplate reader. The measurements were expressed as percentages based on the absorbance measured for the well in which 10% Triton X-100 was added to a final concentration of 0.1% to completely lyse the cells. For a significance test, the measurements were compared with those for the control in which a 10% DMSO solution in DPBS was added. The cells on each well in the 96-well plate were treated with 150.0 μl of MTT with a concentration of 0.5 mg/ml for 4 hours at 37° C. and then, agitated in the presence of 50.0 μl of DMSO, after which a measurement was taken of the absorbance at 570 nm by use of a microplate reader. The absorbance values measured were expressed as percentages based on the absorbance measured of the control which was added with 10% DMSO/DPBS.

Inhibitory Activity Against TNF-α

In order to examine the neuroprotection mechanism of Scutellariae Radix, its inhibitory activity against TNF-α was also measured. BV-2 cells were treated with a Scutellariae Radix extract at different concentrations, in combination with LPS and, after 20 hours of the treatment, a supernatant was obtained. Separately, L929 cells were cultured in 96-well plates and the used medium of each well was replaced with 50 μl a fresh serum-free DMEM. In each well, 50 μl of a medium, 50 μl of rTNF and 50 μl of the supernatant were added and subjected to serial dilution, followed by the addition of 50 μl of DMEM+10% FBS+Actinomycin D (5 μg/ml).

After incubation for 18 hours, each well was deprived of a supernatant. After being added with 100 μl of crystal violet, the cells were washed with flowing water for 10 min and dried. The cells were suspended in 100 μl of 0.5% SDS, vortexed for 30 min and measured for absorbance at 590 nm in a plate reader. For the assay of NO production, advantage was taken of the Griess reaction. In this regard, 50 μl of the Griess reagent was added to each well and allowed to stand for 10 min at room temperature. Absorbance at 550 nm was measured in a plate reader to assay the inhibitory activity of Scutellariae Radix against NO production. The results are given in Table 1, below.

TABLE 1

| | Treatment with | | |
|---|---|---|---|
| | | Sample | Sample |
| | LPS | (10 μg/ml) + | (100 μg/ml) + |
| Assay Subject | (100 ng/ml) | LPS | LPS |
| TNF-α Production (pg/ml) | 78.1 | 39.1 | 9.8 |
| NO Production (μm) | 99.15 | 78.14 | 16.82 |

Immunohistochemistry

The tissue sections with a thickness of 40 μm perfused previously were selected. A cpp 32 antibody was used as a primary antibody for immunostaining while an anti-rabbit antibody was employed as a secondary antibody. The tissue sections were immersed in 0.1 M PBS (pH 7.2) for 5 min, washed twice with Triton-X 100 for 15 min and twice with a mixture of 0.1 M PBS and 0.5% BSA for 15 min, and reacted with the primary antibody overnight at room temperature. After being washed twice with a mixture of 0.1 M PBS and 0.5% BSA, the sections were reacted with the secondary antibody for 60 min. The treatment with a mixture of 0.1 M PBS and 0.5% BSA was repeated twice for 15 min, after which the tissue sections were reacted at a ratio of 50:1 with an ABC (avidin-biotin-peroxidase) conjugate at room temperature for 60 min. Each tissue section, after being washed twice with 0.1 M PBS for 15 min, was reacted with 0.1 M PBS containing 0.05% DAB (3,3′-diaminobenzidine, Sigma U.S.A.) and 0.03% hydrogen peroxide. After color expression, the reaction was ceased by adding 0.1 M PBS to each tissue section which was then prepared into a specimen.

Statistics

To determine the therapeutic effect of the herbal extract, a Student's t-test was used in which each experimental group was compared with the control.

Results

1. Dosage, Body Temperature Influence, and Ischemia Inducing Time Period

In order to examine therapeutic effects according to dosage, the Scutellariae Radix extract was dissolved in various quantities in 0.89% physiological saline and intraperitoneally injected at doses of 250 mg, 500 mg and 1,000 mg per kg of the body weight with a total volume of 2.0 ml. These doses corresponded to 0.73 g/kg, 1.45 g/kg and 2.89 g/kg, respectively, as calculated on the basis of dried Scutellariae Radix weight.

Four rats were forced to undergo ischemia for 5, 10, 20 and 30 min, respectively, to determine the optimal time period of inducing ischemia in rats. After re-perfusion, they were sacrificed to provide hippocampal tissue sections which were then studied for the loss of neurons. Damaged pyramidal cells in a hippocampal CA1 subfield were observed to amount to ¼ of the total number of cells when the ischemia induction was carried out for 10 min, which was thus determined to be optimal for the assay of the medicinal effect of the herb.

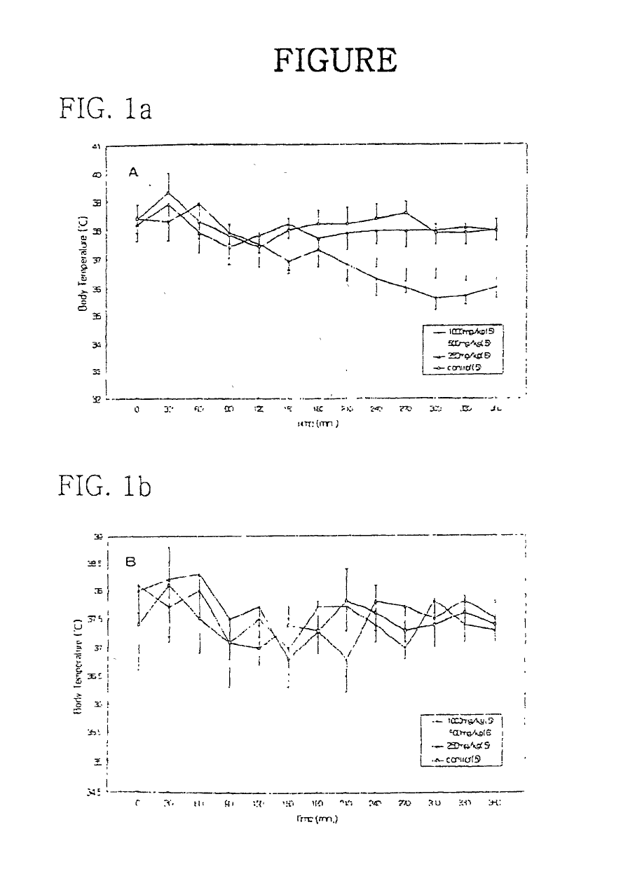

As for the temperature parameter, rats in which cerebral ischemia was induced and re-perfusion was conducted, were monitored for body temperature for 6 hours after the injection of the highest concentration of a sterilized sample thereinto. The results are shown in FIGS. 1a and 1b. The body temperatures plotted in FIG. 1a exhibit hypothermia in the rats into which the Scutellariae Radix extract was injected at various doses after the induction of ischemia. In FIG. 1b, on the other hand, the body temperature of the rats into which the Scutellariae Radix extract was injected at various doses (250, 500 and 1,000 mg/kg) during the transient, global cerebral ischemia were maintained constant (normothermia) (temperature of the rectum: 36.5-37.5° C.). In both cases, a Scutellariae Radix solution in 2.0 ml of 0.89% physiological saline was intraperitoneally injected at doses of 250 mg, 500 mg and 1,000 mg/kg 0 and 90 min after the induction of the global cerebral ischemia and the body temperature was measured in the rectum of the rats. Given in FIGS. 1a and 1b were mean±standard deviation values. The numerals within parentheses mean the numbers of the rats used.

In this course, the Scutellariae Radix-administered groups were observed to have decreased body temperature. The body temperature decrease during the induction of ischemia is known to prevent neuronal cells from being damaged, thereby exhibiting a neuroprotective effect (Busto et al., J. Cereb. Blood Flow Metab. 1987, 7:720-738). After the ischemia induction and re-perfusion, the decrease in body temperature was not observed upon the administration of the Scutellariae Radix at a dose of 250 mg/kg, but was monitored upon the administration of the Scutellariae Radix at doses of 500 and 1,000 mg/kg (FIG. 1a). In this regard, the drug was administered by intraperitoneal injection 0 and 90 min after the ischemia induction. In detail, where the Scutellariae Radix was administered at a dose of 1,000 mg/kg, the body temperature was measured to be 37.6±0.5° C. upon the ischemia induction and 36.9±0.4° C. 2 hours later. Subsequently, the body temperature gradually decreased to 35.1±0.4° C. 6 hours after the ischemia induction, which was lower by about 2.5° C. than the temperature at the time of the ischemia induction (37.7±0.5° C.). In the case of the administration of a dose of 500 mg/kg, the body temperature decreased from 37.7±0.5° C. upon ischemia induction to 37.1±0.3° C. 2 hours later and to 35.9±0.4° C. 6 hours later, a body temperature decrease of about 1.8° C.

To determine whether the neuroprotective effect of the Scutellariae Radix was associated with the body temperature decrease, the body temperature of the rats into which the Scutellariae Radix extract was injected were forcibly maintained constant. That is, the two extract-injected groups which showed a body temperature decrease were forced to maintain their body temperature constant 12 hours after the ischemia induction (FIG. 1b) while the loss of neurons was observed. For the 250 mg/kg-administered group which did not show a significant body temperature decrease, they were maintained in a normothermic state by setting an automatic temperature controller at 37° C. to prevent the body temperature from partially decreasing.

2. Observation of Damaged Neurons

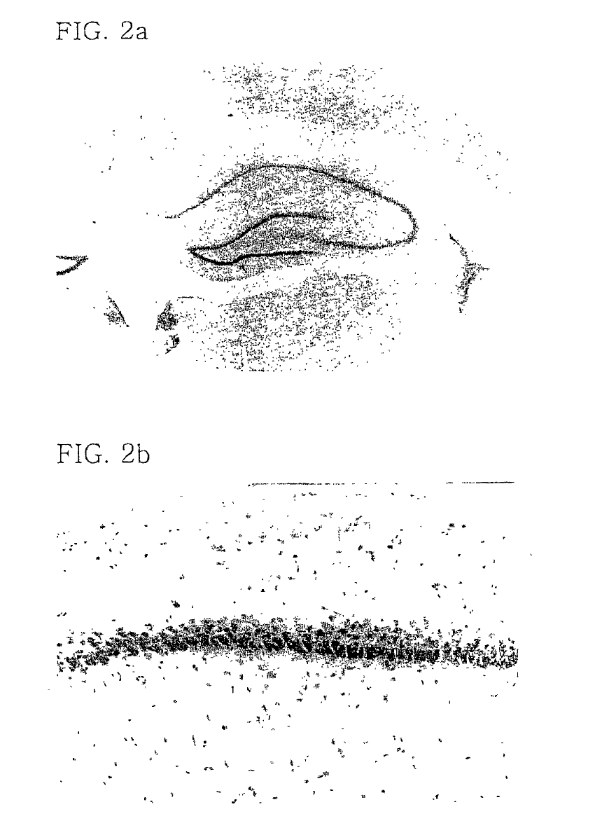

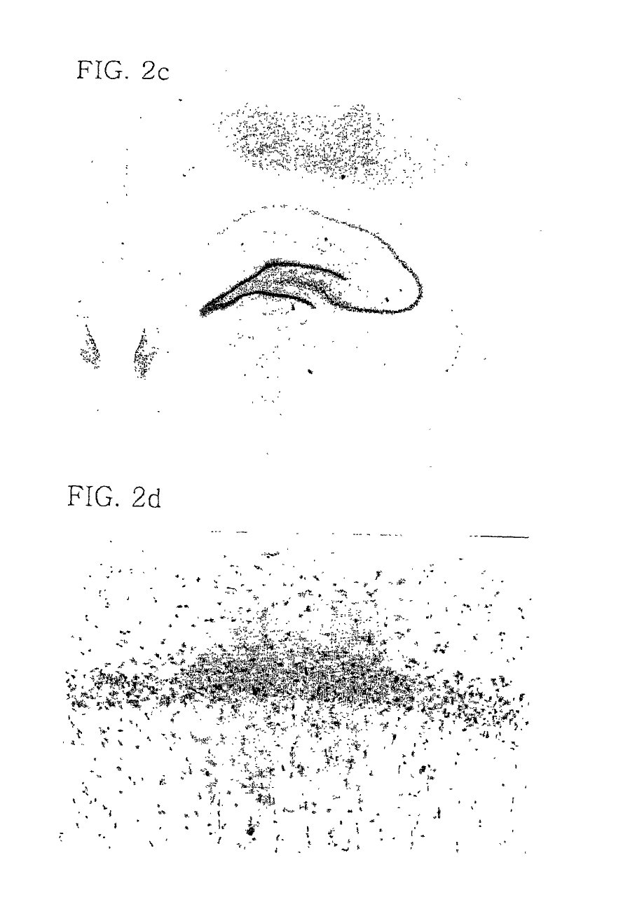

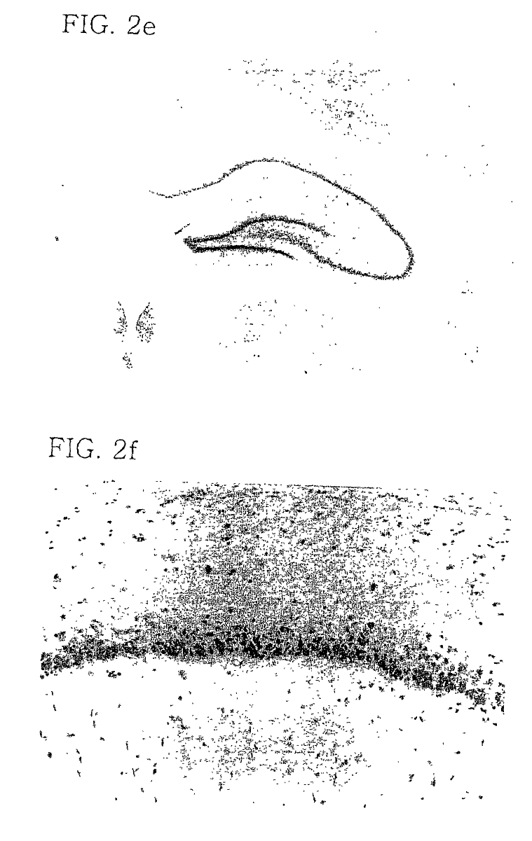

It is reported that, when re-perfusion is conducted after cerebral ischemia caused by 4-vessel occlusion, pyramidal neurons in the hippocampal CA1 subfield are the most susceptible to the ischemia and start to undergo cell death 72 hours after the re-perfusion (Pulsinelli W. A. et al., Ann Neurol 1982, 11:491-498). In this study, the rats were sacrificed one week after the re-perfusion, the time point by which neuronal cells had been completely damaged, and tissue sections obtained from the opposite hippocampus were put under an optical microscope to observe the delayed neuronal death in the hippocampal CA1 subfield. The results are shown in FIGS. 2a to 2f, which are microphotographs of light hippocampuses of rats 7 days after the induction of ischemia for 10 min in the mock group, the control group and the Scutellariae Radix-treated groups.

After coronal suture, brain tissue sections of the dorsal hippocampus were stained with cresyl violet to mark the selective, delayed neuronal loss caused in the hippocampal CA1 subfield by the global ischemia.

In the mock group of FIG. 2a, the arrow denotes the track of CA1 pyramidal neurons. The hippocampal tissue section shown in FIG. 2b is notable in that most pyramidal neutrons in the CA1 subfield have an unchanged (normal) staining pattern. In the control group of FIG. 2c, the stratum pyramidale indicated by the arrow was weakly stained and the neuronal cell damage limitedly occurred within the CA1 subfield. FIG. 2d shows that pyramidal neurons had undergone a coagulative cellular change and were damaged with characteristic apparent gliosis. The groups to which the Scutellariae Radix extract was administered at a dose of 1,000 mg/kg were significantly reduced in the number of the pyramidal neurons in the CA1 field because of their being irrecoverably damaged, as shown in FIGS. 2e and 2f. In FIGS. 2a to 2f, the scale is 100.0 μm long.

In the mock-operated rats which had undergone no ischemia, normal hippocampal neuronal cells were observed in the 490 μm long stratum pyramidale (FIGS. 2a and 2b).

Apoptosis was induced in the control group as shown in FIGS. 2c and 2d. As a rule, once they are induced to undergo apoptosis due to some external or internal stimuli, cells shrink, losing their intrinsic shapes established according to their differentiation. Additionally, the shrinkage breaks the junctions with surrounding cells so that the interaction of the apoptotic cells to adjacent cells is interrupted. As the shrinkage proceeds, apoptotic bodies form while the cell membrane seems to swell like a bulla. In the hippocampal CA1 subfield of the control group to which the physiological saline was administered after the induction of ischemia, neuronal cells were found to undergo apoptosis as detected by the morphological change in FIG. 2d. Unlike the event of FIG. 2b, what FIG. 2d shows is an apoptotic tissue in which cells of interest detach from surrounding cells while the tissue is decomposing. Additionally, the cell bodies of the neuronal cells undergoing apoptosis lose their characteristic pyramidal morphology, forming a kind of single cell. Where the apoptosis was further advanced, the nuclear chromatin was condensed with the nuclear envelope collapsing. In contrast, the neuronal cells in the hippocampal CA1 subfields of the drug-treated groups have a morphology similar to that of normal cells, as apparent in FIGS. 2e and 2f. Herein, because necrotic neurons around the CA1 subfield were very difficult to discriminate from growing microglia, only the viable pyramidal neurons of the CA1 subfield were counted. These cells were easily viewed owing to their perikaryons which healthily extend and their circular nuclei located in the center, being clearly different from neighboring neutrophils. In FIG. 2f, free cells are observed, along with shrunken cell bodies, across the hippocampus, which indirectly demonstrates that the damage was great enough to induce apoptosis. In spite of such great damages a large number of cells were protected from apoptosis, having normal pyramidal morphologies. In addition, the cells were found to retain their junctions to adjacent cells, as before. These results show that the Scutellariae Radix extract can protect neuronal cells of the hippocampal CA1 subfield from the damage caused by 4-vessel occlusion. Although it is not recognized that the Scutellariae Radix extract of the present invention turns the cell cycle of neuronal cells from an apoptotic pathway to a cell survival pathway in what stage of the apoptotic pathway, the Scutellariae Radix extract is identified as being significantly useful in protection from apoptosis (FIGS. 2e and 2f).

3. Protective Effect of Scutellariae Radix Extract on Neuronal Cells

To examine the neuroprotective effect of the Scutellariae Radix extract, it was intraperitoneally injected 0 and 90 min after the induction of cerebral ischemia.

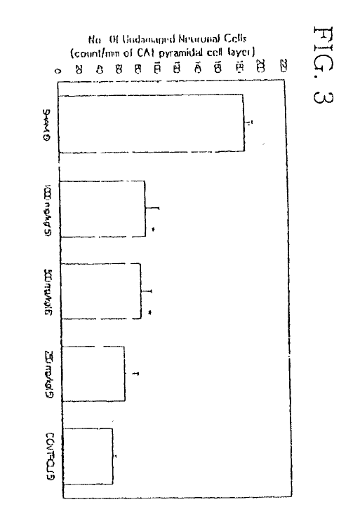

The neuroprotective effects which were seen in the rats whose body temperature was controlled not to decrease below 37° C. (FIG. 1b) are shown in FIG. 3, Zero and 90 min after the induction of ischemia, the Scutellariae Radix extract of the present invention was injected at doses of 250 mg/kg, 500 mg/kg and 1,000 mg/kg. For a control group, 0.89% physiological saline was used at a volume of 2.0 ml/kg. For making the histogram, CA1 pyramidal neurons which were observed to be normal in three hemisphere sections, each having a size of 1×1 mm, were counted and averaged. Numerals within the parentheses stand for the number of experimental animals used. The histogram was plotted on the basis of the mean±standard deviation values. Data from each group was analyzed by use of Student's t-test in which each of the test groups was compared with the control group (*p<0.05).

In the control group to which physiological saline was injected, viable cells were measured to be 47.8±3.1 cells/ml, which is far lower than those in the mock group, measured to be 181±7.9 cells/mm. In the test groups, on the other hand, significant protective effects were brought about as the viable cells were measured to be 84.4±14.7 cells/mm upon injecting the Scutellariae Radix extract at a dose of 1,000 mg/kg and 78.8±11.2 cells/mm upon injecting the Scutellariae Radix extract at a dose of 500 mg/kg (p<0.05). However, a dose of 250 mg/kg did not bring about a significant neuroprotective effect as recognized from the measurement, 61.4±14.8 cells/mm. The neuroprotectively effective doses 1,000 mg/kg and 500 mg/kg, protected the neuronal cells by 27.4% and 23.2%, respectively, greater than did the control group. The difference between the two effective doses is not significant (Table 2, FIG. 3).

TABLE 2
NeuroProtective Effect of Scutellariae Radix Extract on
Cells of CA1 Subfield 7 Days After Performance of 4-VO for
10 min

| | | | SR 1,000 | SR 500 | SR 250 |
|---|---|---|---|---|---|
| | Mock | Control | (mg/kg) | (mg/kg) | (mg/kg) |
| Mean^{a} | 181.5 | 47.8 | 84.4 | 78.8 | 61.4 |
| S.E.M^{a} | 7.9 | 3.1 | 14.7 | 11.2 | 14.8 |
| Count | 6 | 5 | 5 | 6 | 5 |
| P-value | | | 0.033 | 0.019 | 0.208 |
| %^{b} | | 0.0 | 27.4 | 23.2 | 10.2 |

^{a}mean and SEM were expressed as viable cells/mm
^{b}neuroprotective ratio

4. Antioxidative Effects of Scutellariae Radix

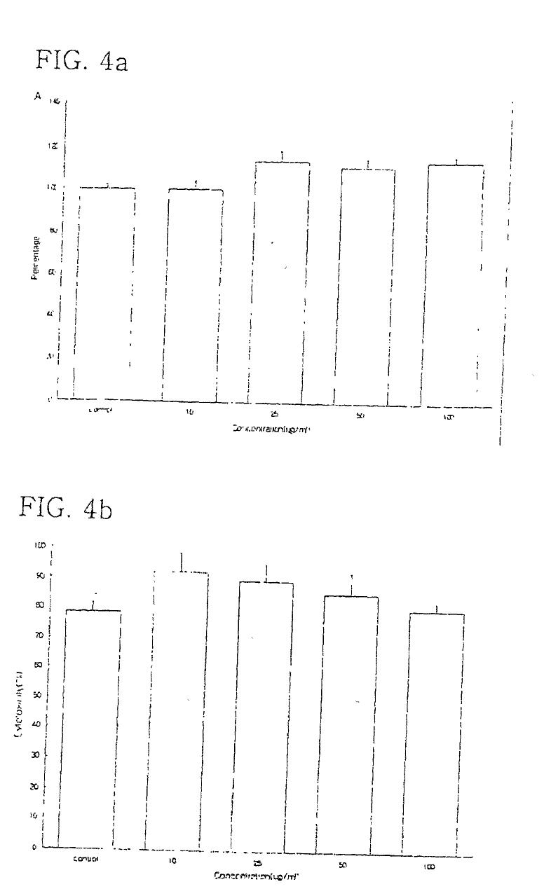

PC12 cells were seeded an amount of 3×10⁴/well and cultured at 37° C. for a time period sufficient to adhered to the wells. After 21 hours of cultivation, the adherent cells were pretreated with a Scutellariae Radix concentration of 10.0 μg/ml, 20.0 μg/ml, 50.0 μg/ml, or 100.0 μg/ml for 3 hours and then cultured in a fresh medium containing 0.5 mM H₂O₂ at 37° C. for 24 hours. The same concentration of the Scutellariae Radix extract as in the pretreatment was also added, together with the fresh medium. After 24 hours of culturing, the cells were analyzed by LDH and MTT assays. The results are given in FIG. 4. A Student's t-test was conducted for comparison to the control group treated with a 10% DMSO/DPBS solution. In FIG. 4a, results of the MTT reduction assay are shown by mean±standard deviation values (n=6). Bars shown in the histogram of FIG. 4b are drawn on the basis of the mean±standard deviation values of percentages of total cell lysis as a result of LDH assays (n=6), *p<0.05, **p<0.01.

When the adherent cells were subjected to oxidative stress for 24 hours in the presence of 0.5 mM H₂O₂, along with the Scutellariae Radix extract with 10.0, 25.0, 50.0 and 100.0 μg/ml after the pretreatment with the Scutellariae Radix extract in the same concentrations 3 hours, the cells were found to exhibit oxidative stress-resistance 113.9% (25.0 μg/ml, p<0.05), 111.1% (50.0 μg/ml, p<0.05) and 113.7% (100.0 μg/ml, p<0.05) higher than that of the control, as measured by an MTT assay (FIG. 4a). However, a significant antioxidative effect was not detected from the Scutellariae Radix extract-treated groups as measured by an LDH assay (FIG. 4b).

When only a drug efficacy is taken into consideration, the neuroprotective effect of the Scutellariae Radix extract is excellent for the following reasons. Drugs for use in the examination of neuro-defense by use of 4-VO models are exemplified mainly by glutamate receptor antagonists, calcium channel antagonists, GABA neurotransmission promoters, NOS inhibitors and antioxidants. Although their efficacies cannot be compared because of the lack of test data thereof, most of these drugs also provide defensive levels of 15-25% as exemplified by LY231617 25-30%, L-NAME 23%, 3-bromo-7-nitroindazole 20%, MK-801 (2 mg/kg, i.p.) 24%, eliprodil (20 mg/kg, i.p.) 25%, NBQX (30 mg/kg, i.p.) 42% 7-NI 17.5%, GYK152466 11% and LY300168 23% (O'Neill M. J., et al., Eur. J. Pharmacol. 1996, 310). The significance of the Scutellariae Radix extract becomes greater in consideration of the fact that herb medicines have very few side-effects since most of them are comprised of combinations of various herbs. Furthermore, when comparing with the known data of other herbs, 16.8% (1,000 mg/kg) and 16.3% (500 mg/kg) for rhubarb, 17.8% (1,200 mg/kg) and 15.6% (600 mg/kg) for Gastrodia edata Bl. 13.7% (1,000 mg/kg) for Magnolia obovata, and 18.4% (1,000 mg/kg) and 16.6% (500 mg/kg) for a mixture of rhubarb, Magnolia obovata, fruits of Poncirus trifoliate Rafin, and Erigeron Canadensis L., the Scutellariae Radix extract of the present invention has remarkably high antioxidant activity.

Conclusion

The observation of the neuroprotective effect of the Scutellariae Radix extract on the forebrain ischemia of rat caused by 4-vessel occlusion allowed the following conclusions to be obtained.

1. Injection of the Scutellariae Radix extract at a dose of 250 mg/kg did not bring about a decrease in body temperature whereas increasing the injection dose to 1,000 mg/kg or 500 mg/kg began to decrease the body temperature from 1 hour after the induction of the cerebral ischemia and maintained the hypothermia up to 6 hours after the induction of the cerebral ischemia.

2. Under the conditions of controlling the body temperature after the induction of cerebral ischemia, the injection of Scutellariae Radix extract at doses of 1,000 mg/kg and 500 mg/kg protected the rats from the damage to neuronal cells upon the induction of cerebral ischemia, at rates 27.4% and 23.2% more efficient, respectively, than no injection of Scutellariae Radix extracts.

3. The neuroprotective effect of the Scutellariae Radix extract was studied for whether it is associated with antioxidation. When cells were under oxidative stress with 0.5 mM H₂O₂ for 24 hours in the presence of the Scutellariae Radix extract in concentrations of 10 μg/ml, 25 μg/ml, 50 μg/ml and 100 μg/ml after the pretreatment with the same concentrations of the extract, significant defensive effects against the cell damage by H₂O₂ were obtained from 25 μg/ml-, 50 μg/ml- and 100 μg/ml-treated groups as measured by an MTT assay. In addition, the Scutellariae Radix extract in concentrations of 10 μg/ml and 100 μg/ml showed significant inhibitory activity against the production of NO.

4. The Scutellariae Radix extract inhibited the production of TNF-α in BV 2 cells in a dose-dependent pattern from 10 μg/ml to 100 μg/ml.

In consequence, it was observed that the Scutellariae Radix extract is be able to make neuronal cells resistant to apoptosis by showing protective activity against the damage to neuronal cells due to the forebrain ischemia caused by 4-vessel occlusion, and inhibitory against the production of TNF-α, as well as antioxidative activity. With these activities, the Scutellariae Radix extract can be used as a neuroprotectant.

### EXAMPLE 3

### Acute Toxicity Test

1. Oral Administration

ICR lineage mice (25±5 weeks old) were divided into 4 groups of ten each, to which the Scutellariae Radix extract was orally administered at doses of 500, 725, 1,000 and 5,000 mg/kg, respectively. The same oral administration was applied to four 10-member groups of Sprague Dawley lineage mice, as well. During the 4 weeks after the oral administration, no mice died in any group. Additionally, there was no difference in appearance between the administered groups and a control group.

2. Peritoneal Administration

ICR lineage mice (25±5 weeks old) were divided into 4 groups of ten each, to which the Scutellariae Radix extract was intraperitoneally injected at doses of 25, 250, 500 and 725 mg/kg, respectively. The same peritoneal injection was performed in four 10-member groups of Sprague Dawley lineage mice, as well. During the 4 weeks after the peritoneal administration, no mice died in any group. Additionally, there was no difference in appearance between the administered groups and a control group.

From the above results, there can be drawn the conclusion that the Scutellariae Radix extract of the present invention has no acute toxicity.

Therefore, the Scutellariae Radix extract of the present invention can be prepared into pharmaceutical preparations suitable for use in the prophylaxis and treatment of nervous system disorders. In this respect, the extract may be formulated with pharmaceutically acceptable expedients or carriers and may be in any form, such as injections, liquid, syrup, pills, capsules, etc.

Depending on the patient's sex, age, body weight and disease severity, the Scutellariae Radix extract of the present invention may be administered at a daily dose of 10 mg to 5,000 mg in one to three installments.

The following preparation examples will further embody the invention.

PREPARATION EXAMPLE 1

| | | |
|---|---|---|
| Scutellariae Radix Extract | 100 | mg |
| Sodium methabisulfite | 3.0 | mg |
| Methyl paraben | 0.8 | mg |
| Propyl paraben | 0.1 | mg |
| Sterile Water for Injection | to 2 | ml |

These ingredients were mixed while adding sterile water to the volume of 2 ml and the solution was filled in a 2 ml ampule to give an injection solution.

These ingredients were mixed while adding sterile water to the volume of 2 ml and the solution was filled in a 2 ml ampule to give an injection solution.

PREPARATION EXAMPLE 2

| | |
|---|---|
| Scutellariae Radix Extract | 200 mg |
| Lactose | 100 mg |
| Starch | 100 mg |
| Magnesium Stearate | proper amount |

These ingredients were mixed and prepared into a tablet.

These ingredients were mixed and prepared into a tablet.

### PREPARATION EXAMPLE 3

Scutellariae Radix Extract 100 mg

Lactose 50 mg

Starch 50 mg

Talc 2 mg

Magnesium Stearate proper amount

These ingredients were mixed and filled in a gelatin capsule in an ordinary manner.

### PREPARATION EXAMPLE 4

Scutellariae Radix Extract 1,000 mg

Sugar 20 g

Isomerized Sugar 20 g

Lemon Flavor proper amount

Sterile Water to 100 ml

These ingredients were mixed in an ordinary manner, filled in a 100 ml brown bottle, and sterilized to give a liquid medicine.

Taken together, the data obtained in the above examples demonstrate that the Scutellariae radix extract of the present invention and its pharmaceutical preparations show high neuroprotective activity with no toxicity, so they

What is claimed is:

1. A Scutellariae Radix extract with neuroprotective activity, prepared by soaking Scutellariae Radix in an extractant, said extractant being selected from water, low alcohols, and mixtures thereof, concentrating the extractant, and freeze-drying the concentrate.

2. A pharmaceutical preparation with neuroprotective activity, comprising a Scutellariae Radix extract as a pharmaceutically effective ingredient in combination with a pharmaceutically acceptable base, said Scutellariae Radix extract being prepared by soaking Scutellariae Radix in an extractant selected from water, low alcohols, and mixtures thereof, concentrating the extractant, and freeze-drying the concentrate.

3. The pharmaceutical preparation as set forth in claim 2, which is used for the prophylaxis and treatment of brain diseases.

4. The pharmaceutical preparation as set forth in claim 2, wherein said brain diseases include apoplexy, Parkinson's disease and senile dementia.
